SEQUENCE LISTING (22 sequences)

SEQ ID NO: 1 - DNA, 21 bp, Artificial Sequence
  Synthetic EIF4G1-F
atttccggtctggttggtctg

SEQ ID NO: 2 - DNA, 22 bp, Artificial Sequence
  Synthetic EIF4G1-R
ccagcaccccctcgattaagaa

SEQ ID NO: 3 - DNA, 20 bp, Artificial Sequence
  Synthetic ELAV1L-F
ggtgacatcgggagaacgaa

SEQ ID NO: 4 - DNA, 20 bp, Artificial Sequence
  Synthetic ELAV1L-R
cccaagctgtgtcctgctac

SEQ ID NO: 5 - DNA, 20 bp, Artificial Sequence
  Synthetic TIA1-F
gagtaacctctggtcagccg

SEQ ID NO: 6 - DNA, 21 bp, Artificial Sequence
  Synthetic TIA1-R
ccgacgtatagagtcttgggc

SEQ ID NO: 7 - DNA, 20 bp, Artificial Sequence
  Synthetic G3BP1-F
ctcagccgcgtaggtttgga

SEQ ID NO: 8 - DNA, 19 bp, Artificial Sequence
  Synthetic G3BP1-R
tctcacaaattcccgcccg

SEQ ID NO: 9 - DNA, 20 bp, Artificial Sequence
  Synthetic PDCD1-F
cagttccaaaccctggtggt

SEQ ID NO: 10 - DNA, 20 bp, Artificial Sequence
  Synthetic PDCD1-R
ggctcctattgtccctcgtg

SEQ ID NO: 11 - DNA, 21 bp, Artificial Sequence
  Synthetic CTLA4-F
tggacacgggactctacatct

SEQ ID NO: 12 - DNA, 19 bp, Artificial Sequence
  Synthetic CTLA4-R
ggcacggttctggatcaat

SEQ ID NO: 13 - DNA, 20 bp, Artificial Sequence
  Synthetic TIM3-F
tgtgcctaacagaggtgtcc

SEQ ID NO: 14 - DNA, 21 bp, Artificial Sequence
  Synthetic TIM3-R
ttccacttctgaggaccttgt

SEQ ID NO: 15 - DNA, 22 bp, Artificial Sequence
  Synthetic LAG3-F
ttggcaatcatcacagtgactc

SEQ ID NO: 16 - DNA, 20 bp, Artificial Sequence
  Synthetic LAG3-R
gctccacacaaagcgttctt

SEQ ID NO: 17 - DNA, 21 bp, Artificial Sequence
  Synthetic CD69-F
ccaccagtccccatttctcaa

SEQ ID NO: 18 - DNA, 21 bp, Artificial Sequence
  Synthetic CD69-R
gtattggcccactgataaggc

SEQ ID NO: 19 - DNA, 20 bp, Artificial Sequence
  Synthetic CD3-F
tgcctcttatcagttggcgt

SEQ ID NO: 20 - DNA, 20 bp, Artificial Sequence
  Synthetic CD3-R
ccaggatactgagggcatgt

SEQ ID NO: 21 - DNA, 20 bp, Artificial Sequence
  Synthetic GAPDH-F
ctcctgttcgacagtcagcc

SEQ ID NO: 22 - DNA, 20 bp, Artificial Sequence
  Synthetic GAPDH-R
ctcctgttcgacagtcagcc

(19) United States
(12) Patent Application Publication
(10) Pub. No.: US 2022/0040183 A1
(43) Pub. Date: Feb. 10, 2022

(54) USE OF INHIBITORS OF STRESS GRANULE FORMATION FOR TARGETING THE REGULATION OF IMMUNE RESPONSES

(71) Applicants: INSERM (Institut National de la Santé et de la Recherche Médicale), Paris (FR); Centre National de la Recherche Scientifique (CNRS), Paris (FR); Université Paul Sabatier Toulouse III, Toulouse (FR)

(72) Inventors: Jean-Jacques FOURNIE, Toulouse Cedex 1 (FR); Don-Marc FRANCHINI, Toulouse Cedex 1 (FR); Olivia LANVIN, Toulouse Cedex 1 (FR); Stefania MILLEVOI, Toulouse Cedex 1 (FR)

(21) Appl. No.: 17/281,408

(22) PCT Filed: Sep. 30, 2019
(86) PCT No.: PCT/EP2019/076414

§ 371 Date: Mar. 30, 2021

(30) Foreign Application Priority Data
Oct. 1, 2018 (EP) 18306286.8

(51) Int. Cl. A61K 31/519; A61K 45/06

(52) U.S. Cl. CPC A61K 31/519; A61K 45/06

(57) ABSTRACT

The mechanisms of tumor escape are numerous, but the immunosuppressive action of coinhibitory molecules has emerged this last decade as the most attractive one for imaging new treatments of cancer. Activation of lymphocytes is indeed regulated by both costimulatory and coinhibitory molecules also called “immune checkpoints”. Now the inventors show that T cell activation triggers mRNA and protein expression of stress granule components and more particularly show that immune checkpoint mRNA interact with said stress granules. More importantly, stress granule inhibitors impair expression of immune checkpoint and thus represent an attractive target for targeting the regulation of immune response.

### FIELD OF THE INVENTION

The present invention relates to the use of inhibitors of stress granule formation for enhancing cytotoxic T lymphocyte-dependent immune responses, in particular, in patients suffering from cancer.

### BACKGROUND OF THE INVENTION

The ability of the immune system to detect and eliminate cancer was first proposed over 100 years ago. Since then, T cells reactive against tumor-associated antigens have been detected in the blood of patients with many different types of cancers, suggesting a role for the immune system in fighting cancer. Innate and adaptive immunity maintains effector cells such as lymphocytes and natural killer cells that distinguish normal cells from “modified” cells as in the case of tumor cells. However, most often tumor cells are able to evade immune recognition and destruction. The mechanisms of tumor escape are numerous, but the immunosuppressive action of coinhibitory molecules has emerged this last decade as the most attractive one for imaging new treatments of cancer. Activation of lymphocytes is indeed regulated by both costimulatory and coinhibitory molecules, some of which belong to the B7/CD28 immunoglobulin superfamily (IgSF), the C-type lectin-like receptor superfamily and the TNF/TNFR superfamily. The balance between these signals determines the lymphocyte activation and consequently regulates the immune response. These costimulatory and coinhibitory molecules were called “immune checkpoints”. Examples of immune checkpoints include B7H3, B7H4, B7H5/VISTA, BTLA, CTLA-4, KIR2DL1-5, KIR3DL1-3, PD-1, PD-L1, PD-L2, CD277, TIM3, LAG3, and TIGIT. Accordingly, the term “immune checkpoint inhibitor” refers to any compound inhibiting the function or expression of an immune checkpoint and typically include peptides, nucleic acid molecules and small molecules, but currently preferred immune checkpoint inhibitors are antibodies. The immune checkpoint inhibitor is administered for enhancing the proliferation, migration, persistence and/or cytotoxic activity of T and NK cells in a subject and in particular the tumor-infiltrating lymphocytes (TIL). One of the most extensively studied immune checkpoint is programmed cell death protein 1 (PD-1) (also known as CD279), which is an IgSF type cell surface receptor expressed by activated T lymphocytes, NK, B cells and macrophages. Its structure comprises an extracellular IgV domain, a transmembrane region and an intracellular tail containing two immunoreceptor tyrosine-based inhibitory motifs (ITIMs). PD-1 is the receptor for PD-L1 expressed by most cell types and PD-L2, so called butyrophilin B7-DC, expressed by various types of myeloid cells. PD-1 engagement by its ligands recruits the intracellular phosphatase Shp2 to dephosphorylate CD28 co-stimulatory molecule, and thus inhibit the activation pathway. This interaction controls autoimmunity, but since PD-L1 or PD-L2 expressions also allow cancer immune evasion, monoclonal antibodies targeting this immunosuppressive receptor preserve the antitumor activity of cytolytic lymphocytes. Hence, the anti-PD-1 nivolumab and pembrolizumab have achieved impressive clinical responses in a sizeable fraction of patients afflicted with solid cancers such as melanoma, non-small-cell lung cancer, or renal-cell carcinoma. Resting T cells do not express PD-1 however, and how activation drives PD-1 expression at the T cell surface remains unknown.

### SUMMARY OF THE INVENTION

The present invention relates to the use of inhibitors of stress granule formation for targeting the regulation of immune responses, in particular, in patients suffering from cancer. In particular, the present invention is defined by the claims.

### DETAILED DESCRIPTION OF THE INVENTION

The object of the present invention relates to a method for targeting the regulation of immune response in a subject in need thereof comprising administering to the subject a therapeutically effective amount of at least one inhibitor of stress granule formation.

More specifically, the present invention provides a method of therapy in subjects in need thereof, comprising administering to the subject a therapeutically effective amount at least one inhibitor of stress granule formation that reduces the expression of an immune checkpoint protein, wherein said administration enhances the proliferation, migration, persistence and/or activity of cytotoxic T lymphocytes (CTLs) in the subject.

More particularly, the present invention provides a method of reducing T cell exhaustion in a subject in need thereof comprising administering to the subject a therapeutically effective amount at least one inhibitor of stress granule formation.

As used herein, the term “cytotoxic T lymphocyte” or “CTL” has its general meaning in the art and refers to a subset of T cells which express CD8 on their surface. CD8 antigens are members of the immunoglobulin supergene family and are associative recognition elements in major histocompatibility complex class I-restricted interactions. They are MHC class I-restricted, and function as cytotoxic T cells. Cytotoxic T lymphocytes are also called, CD8+ T cells, T-killer cells, cytolytic T cells, or killer T cells. The ability of the inhibitor of stress granule formation to enhance proliferation, migration, persistence and/or cytotoxic activity of cytotoxic T lymphocytes may be determined by any assay well known in the art. Typically said assay is an in vitro assay wherein cytotoxic T lymphocytes are brought into contact with target cells (e.g. target cells that are recognized and/or lysed by cytotoxic T lymphocytes). For example, the inhibitor of stress granule formation can be selected for the ability to increase specific lysis by cytotoxic T lymphocytes by more than about 20%, preferably with at least about 30%, at least about 40%, at least about 50%, or more of the specific lysis obtained at the same effector: target cell ratio with cytotoxic T lymphocytes that are contacted by the inhibitor of stress granule formation of the present invention. Examples of protocols for classical cytotoxicity assays are conventional.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include B7-H3, B7-H4, BTLA, CTLA-4, CD277, KIR, PD-1, LAG-3, TIM-3, TIGIT and VISTA. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape. B and T Lymphocyte Attenuator (BTLA), also called CD272, is a ligand of HVEM (Herpesvirus Entry Mediator). Cell surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152 is overexpressed on Treg cells serves to control T cell proliferation. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, short for V-domain Ig suppressor of T cell activation, is primarily expressed on hematopoietic cells so that consistent expression of VISTA on leukocytes within tumors may allow VISTA blockade to be effective across a broad range of solid tumors. As used herein, the term “PD-1” has its general meaning in the art and refers to programmed cell death protein 1 (also known as CD279). PD-1 acts as an immune checkpoint, which upon binding of one of its ligands, PD-L1 or PD-L2, enables Shp2 to dephosphorylate CD28 and inhibits the activation of T cells.

In some embodiments, the inhibitor of stress granule formation is particularly suitable for reducing the expression of PD-1.

As used herein, the term “T cell exhaustion” refers to a state of T cell dysfunction. The T cell exhaustion generally arises during many chronic infections and cancer. T cell exhaustion can be defined by poor effector function, sustained expression of inhibitory receptors, and/or a transcriptional state distinct from that of functional effector or memory T cells. T cell exhaustion generally prevents optimal control of infection and tumors. See, e.g., Wherry E J, Nat Immunol. (2011) 12: 492-499, for additional information about T cell exhaustion. Typically, T cell exhaustion results from the binding of an immune checkpoint protein to at least one of its ligands (e.g. PD1-1 and one of its ligands PD-L1 or PD-L2).

In some embodiments, the subject suffers from a cancer, in particular a colorectal cancer, and the method of the present invention is thus suitable for enhancing the proliferation, migration, persistence and/or cytoxic activity of tumor infiltrating cytotoxic T lymphocytes. As used herein, the term “tumor infiltrating cytotoxic T lymphocyte” refers to the pool of cytotoxic T lymphocytes of the patient that have left the blood stream and have migrated into a tumor. Accordingly, the method of the present invention is particularly suitable for the treatment of cancer.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In particular, the method of the present invention is suitable for the treatment of a cancer characterized by a high tumor infiltration of cytotoxic T lymphocytes that express an immune checkpoint protein. Typically said tumor-infiltration of cytotoxic T lymphocytes is determined by any conventional method in the art. For example, said determination comprises quantifying the density of cytotoxic T lymphocytes that express at least one immune checkpoint protein (e.g. PD-1) in a tumor sample obtained from the patient.

As used herein, the term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumor of the patient or performed in metastatic sample distant from the primary tumor of the patient, for example an endoscopical biopsy performed in the bowel of the patient affected by a colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, through flow cytometry methods and through methods of gene or protein expression analysis, including genomic and proteomic analysis. The tumor tissue sample can, of course, be patiented to a variety of well-known post-collection preparative and storage techniques (e.g., fixation, storage, freezing, etc.). The sample can be fresh, frozen, fixed (e.g., formalin fixed), or embedded (e.g., paraffin embedded). The tumor tissue sample can be used in microarrays, called as tissue microarrays (TMAs). TMA consists of paraffin blocks in which up to 1000 separate tissue cores are assembled in array fashion to allow multiplex histological analysis. This technology allows rapid visualization of molecular targets in tissue specimens at a time, either at the DNA, RNA or protein level. TMA technology is described in WO2004000992, U.S. Pat. No. 8,068,988, Olli et al 2001 Human Molecular Genetics, Tzankov et al 2005, Elsevier; Kononen et al 1198; Nature Medicine.

In some embodiments, the quantification of density of cytotoxic T lymphocytes that express at least one immune checkpoint protein is determined by immunohistochemistry (IHC). For example, the quantification of the density of cytotoxic T lymphocytes is performed by contacting the tissue tumor tissue sample with a binding partner (e.g. an antibody) specific for a cell surface marker of said cells. Typically, the quantification of density of cytotoxic T lymphocytes is performed by contacting the tissue tumor tissue sample with a set of binding partners (e.g. an antibody) specific for CD8 and for the immune checkpoint protein (e.g. PD-1).

Typically, the density of cytotoxic T lymphocytes that express at least one immune checkpoint protein (e.g. PD-1) is expressed as the number of these cells that are counted per one unit of surface area of tissue sample, e.g. as the number of cells that are counted per cm² or mm² of surface area of tumor tissue sample. In some embodiments, the density of cells may also be expressed as the number of cells per one volume unit of sample, e.g. as the number of cells per cm³ of tumor tissue sample. In some embodiments, the density of cells may also consist of the percentage of the specific cells per total cells (set at 100%).

Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated the binding partners. After washing, the labeled antibodies that are bound to a marker of interest are revealed by the appropriate technique, depending of the kind of label being borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 1251) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above described), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In a particular embodiment, quantification of the percentage of cytotoxic T lymphocytes that express at least one immune checkpoint protein (e.g. PD-1) is determined by an automatized microscope which allows measurement of morphometric and fluorescence characteristics in the different cell compartments (membrane/cytoplasm/nuclei) and quantifying preciously the percent of interest cells. Briefly the quantification of percent of cytotoxic T lymphocytes that expression at least one immune checkpoint protein (e.g. PD-1) is performed by following steps: i) providing tissue microarray (TMA) containing RCC samples, ii) TMA samples are stained with anti-CD3, anti-CD8, and anti-PD-1 antibodies, iii) the samples are further stained with an epithelial cell marker to assist in automated segmentation of tumour and stroma, iv) TMA slides are then scanned using a multispectral imaging system, v) the scanned images are processed using an automated image analysis software (e.g. Perkin Elmer Technology) which allows the detection and segmentation of specific tissues through powerful pattern recognition algorithms, a machine-learning algorithm is trained to segment tumor from stroma and identify cells labelled; vi) the percent of cytotoxic T lymphocytes that expression at least one immune checkpoint protein (e.g. PD-1) within the tumour areas is calculated; vii) a pathologist rates lymphocytes percentage; and vii) manual and automated scoring are compared with survival time of the subject.

In some embodiments, the cell density of cytotoxic T lymphocytes is determined in the whole tumor tissue sample, is determined in the invasive margin or centre of the tumor tissue sample or is determined both in the centre and the invasive margin of the tumor tissue sample.

Accordingly a further object of the present invention relates to a method of treating cancer in a patient in need thereof comprising i) quantifying the density of cytotoxic T lymphocytes that express at least one immune checkpoint protein (e.g. PD-1) in a tumor tissue sample obtained from the patient ii) comparing the density quantified at step i) with a predetermined reference value and iii) administering to the patient a therapeutically effective amount of the inhibitor of stress granule formation when the density quantified at step i) is higher than the predetermined reference value.

As used herein, the term “the predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. ROC curve is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the subject suffers from a viral infection. Examples of viral infections treatable by the present invention include those caused by single or double stranded RNA and DNA viruses, which infect animals, humans and plants, such as retroviruses, poxviruses, immunodeficiency virus (HIV) infection, echovirus infection, parvovirus infection, rubella virus infection, papillomaviruses, congenital rubella infection, Epstein-Barr virus infection, mumps, adenovirus, AIDS, chicken pox, cytomegalovirus, dengue, feline leukemia, fowl plague, hepatitis A, hepatitis B, HSV-1, HSV-2, hog cholera, influenza A, influenza B, Japanese encephalitis, measles, parainfluenza, rabies, respiratory syncytial virus, rotavirus, wart, and yellow fever, adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV).

As used herein, the term “stress granule” has its general meaning in the art and refers to an aggregate of proteins and mRNAs that form in a cell under stress conditions. The poly(A)-mRNAs in a stress granule are present in stalled pre-initiation complexes. A stress granule can contain one or more (e.g., two, three, four, or five) of the following proteins/complexes, including but not limited to: 40S ribosomal subunits, eIF4E, eIF4G, eIF4A, eIF4B, poly(A) binding protein (Pabp), eIF3, and eIF2. Additional examples of proteins that can be found in stress granules are described herein. Additional examples of proteins that can be found in stress granules are known in the art (see, for example, Buchan et al., Mol. Cell 36:932, 2009). By the term “stress granule formation” is meant the formation or detection of at least one stress granule in a cell. Stress granule formation in a cell can be detected, for example, by microscopy (e.g., immunofluorescence microscopy) or the detection of phosphorylated eIF2a. Additional methods for detecting stress granule formation in a cell are described herein and are known in the art. As used herein, the expression “inhibitor of stress granule formation” means any compound natural or not that is capable of inhibition of said formation. The inhibitor can be of any nature and include among other small organic molecules, peptides, polypeptides, antibodies, lipids, nucleic acids . . . .

In some embodiments, the inhibitor of the present invention inhibits the activity or expression of a protein, in particular a kinase that is involved in the signalling pathway leading to the formation of stress granule. In some embodiments, the inhibitor of the present invention is an inhibitor of the activity or expression of a kinase selected from the group consisting of GCN2 (e.g. Wek—Mol Cell Biol 1995), PERK (see e.g. Harding—Mol Cell 2000), PKR (e.g. Srivastava—J Biol Chem 1998), HRI (e.g. McEwen—J Biol Chem 2005), mTOR, CK2 (e.g. Reineke—Mol Cell Biol 2017), DYRK3 (e.g. Wippich—Cell 2013), AMPK (e.g. Mahboubi—BBA Mol Cell Res 2015), ROCK1 (e.g. Tsai—Cellular Signalling 2010), S6K1 ((e.g. Sfakianos—Cell Death & Diff 2018), S6K2 (e.g. Sfakianos—Cell Death & Diff 2018) and OGT (e.g. Ohn—Nat Cell Biol 2008). In some embodiments, the inhibitor is an inhibitor of activity or expression of GCN2 or PERK.

As used herein the term ‘GCN2” has its general meaning in the art and refers to the eukaryotic translation initiation factor 2 alpha kinase 4 (Berlanga J J et al. (1999) “Characterization of a mammalian homolog of the GCN2 eukaryotic initiation factor 2alpha kinase”. Eur J Biochem.; 265(2):754-62). Inhibitors of GCN2 activity are well known in the art (Brazeau, Jean-Francois, and Gerard Rosse. “Triazolo [4, 5-d] pyrimidine Derivatives as Inhibitors of GCN2.” (2014): 282-283). In some embodiments, the inhibitor of GCN2 activity is 3-(1H-indazol-6-yl)-N-[1-(oxan-4-yl)pyrazol-4-yl]triazolo[4,5-d]pyrimidin-5-amine (also named as G CN2-IN-1; SCHEMBL15148977; MolPort-044-830-636; ZINC217873341; A-92; or HY-100877). In some embodiments, the inhibitor of GCN2 activity is 3-(4-ethoxyphenyl)-N-[1-(3-piperidin-4-ylpropyl)pyrazol-4-yl]triazolo[4,5-d]pyrimidin-5-amine.

As used herein the term “PERK” has its general meaning in the art and refers to the eukaryotic translation initiation factor 2-alpha kinase 3 (Shi Y, et al. (1998) “Identification and characterization of pancreatic eukaryotic initiation factor 2 alpha-subunit kinase, PEK, involved in translational control”. Mol Cell Biol. 18(12):7499-509). Inhibitors of PERK activity are well known in the art (Axten, Jeffrey M. “Protein kinase R (PKR)-like endoplasmic reticulum kinase (PERK) inhibitors: a patent review (2010-2015).” Expert opinion on therapeutic patents 27.1 (2017): 37-48). Suitable inhibitors of PERK activity include those disclosed in WO2015/056180 and WO2014/161808. In some embodiments, the inhibitor of PERK activity is 1-[5-(4-Amino-7-methyl-7H-pyrrolo[2,3-d]pyrimidin-5-yl)indolin-1-yl]-2-(3-trifluoromethylphenyl)ethanone. In some embodiments, the inhibitor of PERK activity is 1-[5-(4-amino-7-methylpyrrolo[2,3-d]pyrimidin-5-yl)-2,3-dihydroindol-1-yl]-2-[3-(trifluoromethyl)phenyl]ethanone (also named as GSK2606414).

In some embodiments, the inhibitor of the present invention inhibits the activity or expression of a protein that is structurally involved in formation of stress granule. In some embodiments, the inhibitor of the present invention is an inhibitor of the activity or expression of a protein selected from the group consisting of ABCF1, ADAR, ADD1, AGO1, AGO2, AHSA1, AKAP9, ALYREF, ANG, APOBEC3G, AQR, ATP2C1, ATXN2, ATXN2L, BCCIP, BRF1, CALR, CAPRIN1, CASC3, CCAR1, CCDC124, CCR4, CDC37, CELF1, CELF2, CIRBP, CNBP, CNOT8, CPEB1, CPEB2, CPEB3, CPEB4, CYFIP2, DAZAP1, DAZAP2, DAZL, DCP1A, DCP1B, DCP2, DDX1, DDX39A, DDX39B, DDX3X, DDX3Y, DDX5, DDX58, DDX6, DHX30, DHX33, DHX36, DHX58, DHX9, DROSHA, DYRK3, EDC3, EDC4, EIF2A, EIF2AK2, EIF2C1, EIF2S1, EIF2S2, EIF2S3, EIF3A, EIF3B, EIF3C, EIF3D, EIF3E, EIF3F, EIF3G, EIF3H, EIF3I, EIF3J, EIF3K, EIF3L, EIF3M, EIF4A1, EIF4A2, EIF4A3, EIF4B, EIF4E, EIF4G1, EIF4G2, EIF4G3, EIF4H, EIF5, EIF5A, EIF5A2, EIF5B, ELAVL1, ELAVL2, ELAVL3, ELAVL4, ETF1, EWSR1, FAM120A, FASTK, FMR1, FUBP1, FUBP3, FUS, FXR1, FXR2, G3BP1, G3BP2, GBP2, GIGYF2, GRB7, GSPT1, GSPT2, HDAC6, HNRNPA0, HNRNPA1, HNRNPA2B1, HNRNPA3, HNRNPAB, HNRNPC, HNRNPD, HNRNPH1, HNRNPK, HNRNPL, HNRNPM, HNRNPR, HNRNPU, HOPX, HSP90AA1, HSPA8, HSPB1, HSPD1, HTT, IGF2BP1, IGF2BP2, IGF2BP3, ILF2, ILF3, IPO8, KHDRBS1, KHSRP, KPNA2, KPNA4, KPNA5, KPNB1, LARP4, LARP4B, LIN28A, LIN28B, LSM1, LSM12, LSM14A, LSM14B, MAP1LC3A, MAPK8, MATR3, MBNL1, MCRIP1, MCRIP2, METAP2, MEX3A, MEX3B, MSI1, MSI2, NCL, NELFE, NKRF, NOLC1, NONO, NPM1, NRG2, NUFIP2, NXF1, NXF5, OAS1, OAS2, OAS3, OGFOD1, OGG1, OGN, PABPC1, PABPC3, PABPC4, PABPC5, PAN2, PAN3, PARN, PATL1, PCBP1, PCBP2, PFN1, PFN2, PHB2, PKP1, PKP3, PNPT1, PPP1R8, PQBP1, PRKCA, PRKRA, PRMT1, PRRC2C, PSD3, PSPC1, PTBP1, PTK2, PUM1, PUM2, PURA, PURB, QKI, RACK1, RAN, RBM15, RBM17, RBM25, RBM3, RBM4, RBM42, RC3H1, RECQL, RHOA, RNASEL, RNH1, ROCK1, RPL3, RPS11, RPS18, RPS19, RPS24, RPS3, RPS6, RPS6KA3, RTCA, SAFB2, SAMD4A, SERBP1, SF1, SFPQ, SLBP, SMG1, SMN1, SMN2, SND1, SPATS2L, SRP68, SRSF5, SRSF7, SRSF9, STAU1, STAU2, SYNCRIP, TAF15, TARDBP, TDRD3, TIA1, TIAL1, TNPO1, TNRC6A, TNRC6B, TRAF2, TRIM2, TRIM3, TRIP6, TROVE2, UBAP2L, UPF1, UPF2, UPF3A, UPF3B, USP10, USP6, UTP18, WDR62, XRN1, XRN2, YBX1, YBX3, YTHDF1, YTHDF2, ZBP1, ZC3H11A, ZC3HAV1, ZFP36, and ZONAB.

As used herein, the term “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of targeted protein mRNA by binding thereto and thus preventing protein translation or increasing mRNA degradation, thus decreasing the level of targeted protein, and thus activity, in a cell. For example, antisense oligonucleotides of at least about 15 bases and complementary to unique regions of the mRNA transcript sequence encoding targeted protein can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. targeted protein gene expression can be reduced by contacting a subject or cell with a small double stranded RNA (dsRNA), or a vector or construct causing the production of a small double stranded RNA, such that targeted protein gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing targeted protein. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to, nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Ban viruses; papilloma viruses; herpes virus; vaccinia virus; polio virus; and RNA virus such as a retrovirus. One can readily employ other vectors not named but known to the art.

According to the invention, the inhibitor is administered to the patient in a therapeutically effective amount. By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the inhibitor of the present invention is administered to the subject in combination with at least one immune checkpoint inhibitor. Examples of immune checkpoint inhibitor includes PD-1 antagonists, PD-L1 antagonists, PD-L2 antagonists, CTLA-4 antagonists, VISTA antagonists, TIM-3 antagonists, LAG-3 antagonists, IDO antagonists, KIR2D antagonists, A2AR antagonists, B7-H3 antagonists, B7-H4 antagonists, and BTLA antagonists.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.570, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signalling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

Typically the active ingredient of the present invention (e.g. the inhibitor) is combined with pharmaceutically acceptable excipients, and optionally sustained-release matrices, such as biodegradable polymers, to form pharmaceutical compositions. The term “Pharmaceutical” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

A further object of the present invention relates to an in vitro or ex vivo method of reducing the expression of at least one immune checkpoint protein in a population of T cells comprising contacting the population of T cells with an amount of at least one inhibitor of stress granule formation.

As used herein, the term “T cells” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8+ T cells, CD4+ T cells, Gamma delta T cells, and Tregs.

In some embodiments, the population of T cells is a population of cytotoxic T lymphocytes (as defined above). Naive CD8+ T cells have numerous acknowledged biomarkers known in the art. These include CD45RA+CCR7+HLA-DR−CD8+ and the TCR chain is formed of alpha chain (α) and beta chain (β). Persisting (central memory and effector memory), non-persisting (effector or exhausted subpopulations), anergic/tolerant and senescent regulatory CD8+ T cells can be discriminated on their differential expression of surface markers including (but not limited to) CCR7, CD44, CD62L, CD122; CD127; IL15R, KLRG1, CD57, CD137, CD45RO, CD95, PD-1 CTLA, Lag3 and transcription factors such as T-bet/Eomes, BCL6, Blimp-1, STAT3/4/5 ID2/3, NFAT, FoxP3.

In some embodiments, the population of T cells is a population of CD4+ T cells. The term “CD4+ T cells” (also called T helper cells or TH cells) refers to T cells which express the CD4 glycoprotein on their surfaces and which assist other white blood cells in immunologic processes, including maturation of B cells into plasma cells and memory B cells, and activation of cytotoxic T cells and macrophages. CD4+ T cells become activated when they are presented with peptide antigens by MHC class II molecules, which are expressed on the surface of antigen-presenting cells (APCs). Once activated, they divide rapidly and secrete cytokines that regulate or assist in the active immune response. These cells can differentiate into one of several subtypes, including TH1, TH2, TH3, TH17, TH9, TFH or Treg, which secrete different cytokines to facilitate different types of immune responses. Signaling from the APC directs T cells into particular subtypes. In addition to CD4, the TH cell surface biomarkers known in the art include CXCR3 (Th1), CCR4, Crth2 (Th2), CCR6 (Th17), CXCR5 (Tfh) and as well as subtype-specific expression of cytokines and transcription factors including T-bet, GATA3, EOMES, RORγT, BCL6 and FoxP3.

In some embodiments, the population of T cells is a population of gamma delta T cells. Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MHC molecules of antigen-presenting cells. Gamma 9 delta 2 T cells (sometimes also called gamma 2 delta 2 T cells) are gamma delta T cells bearing TCR receptors with the variable domains Vγ9 and Vδ2. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62: 1-5) or by other intracellular parasites, such as mycobacteria (Constant et al., Infection and Immunity, December 1995, vol. 63, no. 12: 4628-4633) or protozoa (Behr et al., Infection and Immunity, 1996, vol. 64, no. 8: 2892-2896). They may also be cancer cells (Poccia et al., J. Immunol., 159: 6009-6015; Fournie and Bonneville, Res. Immunol., 66th Forum in Immunology, 147: 338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

In some embodiments, the population of T cells is a population of CAR-T cells. As used herein the term “CAR-T cell” refers to a T lymphocyte that has been genetically engineered to express a CAR. The definition of CAR T-cells encompasses all classes and subclasses of T-lymphocytes including CD4+, CD8+ T cells, gamma delta T cells as well as effector T cells, memory T cells, regulatory T cells, and the like. The T lymphocytes that are genetically modified may be “derived” or “obtained” from the subject who will receive the treatment using the genetically modified T cells or they may “derived” or “obtained” from a different subject. The term “chimeric antigen receptors (CARs),” as used herein, may refer to artificial T-cell receptors T-bodies, single-chain immunoreceptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In some embodiments, CARs comprise an intracellular activation domain, a transmembrane domain, and an extracellular domain that may vary in length and comprises a tumor associated antigen binding region. In particular aspects, CARs comprise fusions of single-chain variable fragments (scFv) derived from monoclonal antibodies, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

In some embodiments, the population of T cells is specific for an antigen. The term “antigen” (“Ag”) as used herein refers to protein, peptide, nucleic acid or tissue or cell preparations capable of eliciting a T cell response. In some embodiments, the antigen is a tumor-associated antigen (TAA). Examples of TAAs include, without limitation, melanoma-associated Ags (Melan-A/MART-1, MAGE-1, MAGE-3, TRP-2, melanosomal membrane glycoprotein gp100, gp75 and MUC-1 (mucin-1) associated with melanoma); CEA (carcinoembryonic antigen) which can be associated, e.g., with ovarian, melanoma or colon cancers; folate receptor alpha expressed by ovarian carcinoma; free human chorionic gonadotropin beta (hCGP) subunit expressed by many different tumors, including but not limited to ovarian tumors, testicular tumors and myeloma; HER-2/neu associated with breast cancer; encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B-cell lymphoma that can generate tumor-specific immunity (attributed to idiotype-specific humoral immune response), Mesothelin associated with pancreatic, ovarian and lung cancer, P53 associated with ovarian, colorectal, non small cell lung cancer, NY-ESO-1 associated with testis, ovarian cancer, EphA2 associated with breast, prostate, lung cancer, EphA3 associated with colorectal carcinoma, Survivin associated with lung, breast, pancreatic, ovarian cancer, HPV E6 and E7 associated with cervical cancer, EGFR associated with NSCL cancer. Moreover, Ags of human T cell leukemia virus type 1 have been shown to induce specific cytotoxic T cell responses and anti-tumor immunity against the virus-induced human adult T-cell leukemia (ATL). Other leukemia Ags can equally be used. Tumor-associated antigens which can be used in the present invention are disclosed in the book “Categories of Tumor Antigens” (Hassane M. et al Holland-Frei Cancer Medicine (2003). 6th edition.) and the review Gregory T. et al (“Novel cancer antigens for personalized immunotherapies: latest evidence and clinical potential” Ther Adv Med Oncol. 2016; 8(1): 4-31) all of which are herein incorporated by reference. In some embodiments, the tumor-associated antigen is melanoma-associated Ags.

Typically, the population of T cells is prepared from a PBMC. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Cord blood mononuclear cells are further included in this definition. Typically, the PBMC sample according to the invention has not been subjected to a selection step to contain only adherent PBMC (which consist essentially of >90% monocytes) or non-adherent PBMC (which contain T cells, B cells, natural killer (NK) cells, NK T cells and DC precursors). A PBMC sample according to the invention therefore contains lymphocytes (B cells, T cells, NK cells, NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer, which will preferentially lyse red blood cells. Such procedures are known by a skilled person in the art. For example, the initial cell preparation consists of PBMCs from fresh or frozen (cytopheresed) blood. Isolated T cell (or APC) can be analysed in flux cytometry. Several doses of the T cells (or APC) cellular product can be manufactured from one frozen cytopheresis. Typically, 100 million frozen PBMCs from cytopheresis yield 1 to 5 billion cells with the classical method of preparation. Standard methods for purifying and isolating T cells are well known in the art. For instance, cell sorting is a current protocol that may be used to isolate and purify the obtained CTLs. Typically, multimers (e.g. tetramers or pentamers) consisting of MHC class 1 molecules loaded with the immunogenic peptide are used. To produce multimers, the carboxyl terminus of an MHC molecule, such as, for example, the HLA A2 heavy chain, is associated with a specific peptide epitope, and treated so as to form a multimer complex having bound hereto a suitable reporter molecule, preferably a fluorochrome such as, for example, fluoroscein isothiocyanate (FITC), phycoerythrin, phycocyanin or allophycocyanin. The multimers produced bind to the distinct set of CD8+ T cell receptors (TcRs) on a subset of CD8+ T cells to which the peptide is MHC class I restricted. Following binding, and washing of the T cells to remove unbound or non-specifically bound multimer, the number of CD8+ cells binding specifically to the HLA-peptide multimer may be quantified by standard flow cytometry methods, such as, for example, using a FACS Calibur Flow cytometer (Becton Dickinson). The multimers can also be attached to paramagnetic particles or magnetic beads to facilitate removal of non-specifically bound reporter and cell sorting. Such particles are readily available from commercial sources (eg. Beckman Coulter, Inc., San Diego, Calif., USA).

In some embodiments, once the selected naive T cells (e.g. naive CD8+ T cells) are purified they are subsequently admixed and incubated the population of antigen presenting cells (APCs) for a time sufficient to activate and enrich for a desired population of activated T cells, such as activated helper T cells, and preferably, CTLs or CD8+ memory T cells. Such activated T cells preferably are activated in a peptide-specific manner. The ratio of substantially separated naive T cells to APCs may be optimized for the particular individual, e.g., in light of individual characteristics such as the amenability of the individual's lymphocytes to culturing conditions and the nature and severity of the disease or other condition being treated. Any culture medium suitable for growth, survival and differentiation of T cells is used for the coculturing step. Typically, the base medium can be RPMI 1640, DMEM, IMDM, X-VIVO or AIM-V medium, all of which are commercially available standard media. Typically, the naive T cells are contacted with the APCs of the present invention for a sufficient time to activate a CTL response. In some embodiments, one or more selected cytokines that promote activated T cell growth, proliferation, and/or differentiation are added to the culture medium. The selection of appropriate cytokines will depend on the desired phenotype of the activated T cells that will ultimately comprise the therapeutic composition or cell therapy product. For instance cytokines include IL-1, IL-2, IL-7, IL-4, IL-5, IL-6, IL-12, IFN-γ, and TNF-α. In some embodiments, the culture medium comprises antibodies. Exemplary antibodies include monoclonal anti-CD3 antibodies, such as that marked as ORTHOCLONE OKT®3 (muromonab-CD3).

In some embodiments, the population of T cells is contacted with the inhibitor of stress granule formation for a time sufficient for to reduce the expression of checkpoint proteins. For instance, the population of T cells and the inhibitor of stress granule formation are contacted with each other for 1, 2, 3, 4, 5, 6, 7, 8, 9, 10, 11, 12, 13, 14, 15, 16, 17, 18, 19, 20, 21, 22, 23, 24, 25, 26, 27, 28 or 30 hours. Typically, the inhibitor of stress granule formation is added in the culture medium where the population of T cells is cultured. In some embodiments, the inhibitor of stress granule formation is added when the population of T cells is activated (for instance in presence of a population of APC).

Once the population of T cells is obtained, functionality of the cells may be evaluated according to any standard method which typically include a cytotoxic assay. Cell surface phenotype of the cells with the appropriate binding partners can also be confirmed. Quantifying the secretion of various cytokines may also be performed. Methods for quantifying secretion of a cytokine in a sample are well known in the art. For example, any immunological method such as but not limited to ELISA, multiplex strategies, ELISPOT, immunochromatography techniques, proteomic methods, Western blotting, FACS, or Radioimmunoassays may be applicable to the present invention.

The population of T cells obtained by the method of the present invention may find various applications. More particularly, the population of T cells is suitable for the adoptive immunotherapy. The in vitro or ex vivo method of the present invention is particularly suitable for preventing T cell exhaustion when the population of T cells is administered to a patient for adoptive immunotherapy. As used herein, the term “adoptive immunotherapy” refers the administration of donor or autologous T lymphocytes for the treatment of a disease or disease condition, wherein the disease or disease condition results in an insufficient or inadequate immune response. Adoptive immunotherapy is an appropriate treatment for any disease or disease condition where the elimination of infected or transformed cells has been demonstrated to be achieved by a specific population of T cells. Exemplary diseases, disorders, or conditions that may be treated with the population of T cells as prepared according to the present invention include, for example, include immune disorders, such as immune deficiency disorders, autoimmune disorders, and disorders involving a compromised, insufficient, or ineffective immune system or immune system response; infections, such as viral infections, bacterial infections, mycoplasma infections, fungal infections, and parasitic infections; and cancers.

The invention will be further illustrated by the following figures and examples. However, these examples and figures should not be interpreted in any way as limiting the scope of the present invention.

### FIGURES

FIG. 1. T cell activation triggers mRNA expression of stress granules components. qRT-PCR quantification of stress granules components mRNA at different time points after T cell activation (n=3, means+/−s.d.).

FIG. 2. T cell activation triggers protein expression of stress granules components. Representative western-blot of G3BP1, β-Actin and GAPDH protein expression at different time points after T cell activation (representative of n=3).

FIG. 3. Immune checkpoints mRNA interact with stress granules. qRT-PCR quantification of PDCD1, CTLA4, TIM3, LAG3, CD69 and CD3E mRNAs after immunoprecipitation with an anti-G3BP1 or an isotype (Ig) control antibody from lysates of activated T lymphocytes (n=3, means+/−s.d.).

FIG. 4. Stress granules inhibitors impair immune checkpoints expression. PD-1, LAG3 and TIM3 expression by CD3+ T cells, stimulated for 3 days, with or without A-92 (1 μM) or GSK2606414 (GSK, 10 μM). Two-tailed paired t-test, ** P<0.01, *** P<0.001.

FIG. 5. Stress granules inhibitors impair immune checkpoints expression simultaneously. Contour plots of co-expression profile of PD-1, LAG3 and TIM3 immune checkpoint receptors on activated CD3+ T cells from healthy donors PBMC, treated or not with A-92 (1 μM) or GSK2606414 (GSK, 10 μM). Representative of n=6 experiments.

### EXAMPLE

Methods

Cells.

PBMC were obtained from human healthy donors (Etablissement Français du Sang, Toulouse, France) after Ficoll-Hypaque (GE Healthcare) density centrifugation and cultured in RPMI 1640 medium (ThermoFisher Scientific) supplemented with 10% fetal bovine serum (ThermoFisher Scientific) and L-glutamine (SIGMA Aldrich).

Cell-Based Assay of Immune Checkpoint Expression by T Lymphocytes.

PBMC isolated from healthy donors were activated with CD3/CD28 antibodies-coated beads (ThermoFisher Scientific). When drugs were used (A-92 (1 μM) or GSK2606414 (GSK, 10 μM)), they were added simultaneously to the stimulation. After 3 days of in vitro culture, cells were processed for qRT-PCR, immunoblotting or flow cytometry analysis.

Reverse Transcription and Quantitative Real-Time PCR (qRT-PCR).

After several washes, cells were resuspended in Trizol reagent (ThermoFisher Scientific) and RNA was extracted using the Direct-zol RNA MiniPrep (Zymo Research). RNA reverse-transcription was performed using the SuperScript™ III Reverse Transcriptase Kit (ThermoFischer Scientific) according to the manufacturer's instruction. qRT-PCR were carried out with the ABI PRISM 7500 Real-Time PCR System (Applied Biosystems) with the PowerUp SYBR Green Master Mix (ThermoFischer Scientific) with the primers EIF4G1-F, 5′-ATTTCCGGTCTGGTTGGTCTG-3′ (SEQ ID NO: 1) and EIF4G1-R, 5′-CCAGCACCCCCTCGATTAAGAA-3′ (SEQ ID NO: 2); ELAV1L-F, 5′-GGTGACATCGGGAGAACGAA-3′ (SEQ ID NO: 3) and ELAV1L-R, 5′-CCCAAGCTGTGTCCTGCTAC-3′ (SEQ ID NO: 4); TIA1-F, 5′-GAGTAACCTCTGGTCAGCCG-3′ (SEQ ID NO: 5) and TIA1-R, 5′-CCGACGTATAGAGTCTTGGGC-3′ (SEQ ID NO: 6); G3BP1-F, 5′-CTCAGCCGCGTAGGTTTGGA-3′ (SEQ ID NO: 7) and G3BP1-R, 5′-TCTCACAAATTCCCGCCCG-3′ (SEQ ID NO: 8); PDCD1-F, 5′-CAGTTCCAAACCCTGGTGGT-3′ (SEQ ID NO: 9) and PDCD1-R, 5′-GGCTCCTATTGTCCCTCGTG-3′ (SEQ ID NO: 10); CTLA4-F, 5′-TGGACACGGGACTCTACATCT-3′ (SEQ ID NO: 11) and CTLA4-R, 5′-GGCACGGTTCTGGATCAAT-3′ (SEQ ID NO: 12); TIM3-F, 5′-TGTGCCTAACAGAGGTGTCC-3′ (SEQ ID NO: 13) and TIM3-R, 5′-TTCCACTTCTGAGGACCTTGT-3′ (SEQ ID NO: 14); LAG3-F, 5′-TTGGCAATCATCACAGTGACTC-3′ (SEQ ID NO: 15) and LAG3-R, 5′-GCTCCACACAAAGCGTTCTT-3′ (SEQ ID NO: 16); CD69-F, 5′-CCACCAGTCCCCATTTCTCAA-3′ (SEQ ID NO: 17) and CD69-R, 5′-GTATTGGCCCACTGATAAGGC-3′ (SEQ ID NO: 18); CD3-F, 5′-TGCCTCTTATCAGTTGGCGT-3′ (SEQ ID NO: 19) and CD3-R, 5′-CCAGGATACTGAGGGCATGT-3′ (SEQ ID NO: 20) or GAPDH-F, 5′-CTCCTGTTCGACAGTCAGCC-3′ (SEQ ID NO: 21) and GAPDH-R 5′-CTCCTGTTCGACAGTCAGCC-3′ (SEQ ID NO: 22). GAPDH were used as reference gene. The amplification fold change was calculated with the MET method.

Immunoblotting.

Cells were lysed on ice for 30 min with lysis buffer containing 10 mM Hepes, 100 mM KCl, 150 mM NaCl, 0.5% NP40, 1 mM DTT and a cocktail of protease inhibitors (Complete, Roche). Whole cell lysate was centrifuged at 10.000×g for 15 min at 4° C. Supernatants were collected and cell extract was quantified using the BCA assay. Samples were heated at 95° C. for 5 min in SDS-buffer, separated by SDS-PAGE, and transferred to nitrocellulose membranes. Membranes were blocked in 5% BSA in 1×PBS+0.1% tween-20, probed with the specified antibodies, and detected with HRP based enhanced chemiluminescence (ECL prime western blotting detection reagent, GE Healthcare). Protein expression level was controlled with β-actin and GAPDH.

Immunofluorescence Staining.

Slides with resting or stimulated T lymphocytes were washed and incubated with a blocking solution (10% goat serum in PBS 1X) for 30 min at RT. Immunofluorescence was then performed using a mouse monoclonal anti-G3BP1 primary antibodies (Abcam, ab56574) in combination with 2% goat serum in PBS 1X and incubated overnight at 4° C. Secondary antibodies (Alexa Fluor 488 goat anti-mouse, ThermoFisher Scientific) was added the next day. Coverslips were mounted with Dako fluorescent mounting medium (Dako, Agilent). Images of the stained cells were taken using a Zeiss LSM 780 Axio Observer Z1 confocal microscope.

RNA Immunoprecipitation (RIP).

RNA immuno-precipitation was performed following the protocols described in (Keene et al., 2006; Peritz et al., 2006). Briefly, cell extract was produced from activated CD3+ T lymphocytes isolated from human PBMC of healthy donors with polysomal lysis buffer (10 mM HEPES pH 7.0, 100 mM KCL, 5 mM MgCl2, 0.5% NP40, 1 mM DTT, 80 U RNase OUT (ThermoFisher Scientific) and protease Inhibitor cocktail (Roche)). Protein A/G PLUS agarose beads (20 μl of slurry beads per μg of antibody) were coated with specific or control anti-Ig antibody (3 μg of antibody per mg of extract, per sample). The cell lysate (3 mg of protein) was diluted in the NT2 buffer (50 mM Tris-HCl pH 7.4, 150 mM NaCl, 1 mM MgCl2, 0.05% NP40) and incubated with antibody-coated beads, supplemented with 200 U RNase OUT per sample. 1/100e of the supernatant was kept as input for qRT-PCR analysis. After several washes, the beads were suspended in Trizol reagent (ThermoFisher Scientific), RNA was extracted then processed to reverse transcription and qRT-PCR analysis.

Results

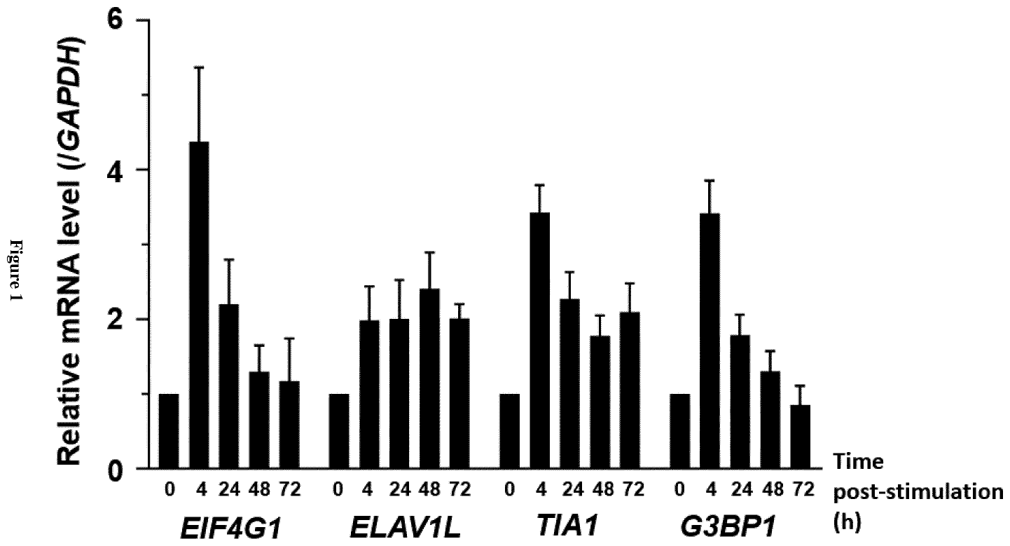

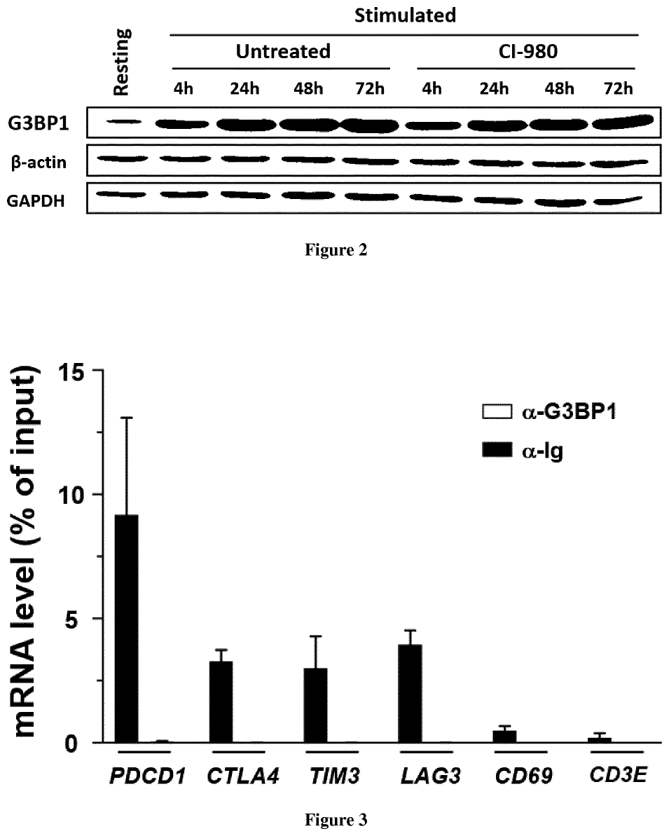

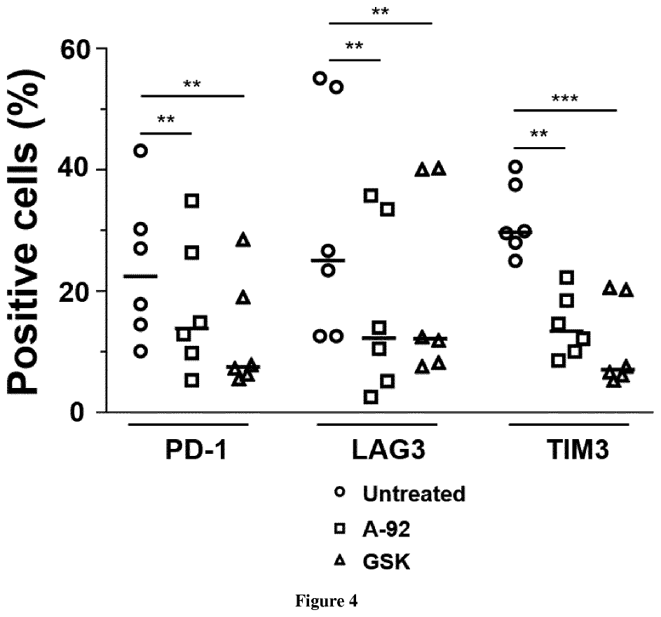

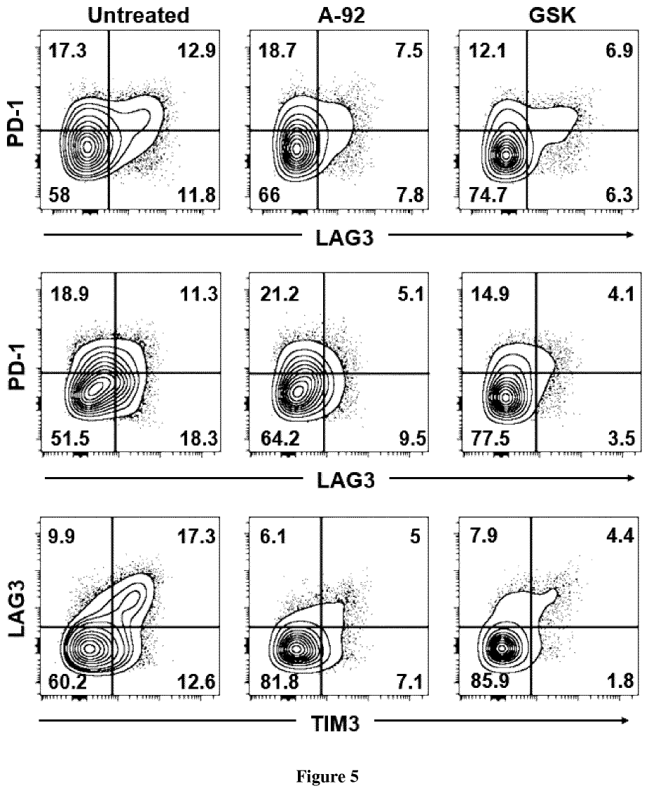

The results are represented in FIGS. 1 to 5. In particular we show that T cell activation triggers mRNA and protein expression of stress granule components (FIGS. 1 and 2). We show that immune checkpoint mRNA interact with stress granule (FIG. 3). More importantly, stress granule inhibitors impair expression of immune checkpoint (FIGS. 4 and 5).

### REFERENCES

Throughout this application, various references describe the state of the art to which this invention pertains. The disclosures of these references are hereby incorporated by reference into the present disclosure.

1. A method for regulating an immune response in a subject in need thereof comprising administering to the subject a therapeutically effective amount of at least one inhibitor of stress granule formation sufficient to regulate the immune response.

2. A method of enhancing proliferation, migration, persistence and/or activity of cytotoxic T lymphocytes (CTLs) in a subject in need thereof, comprising administering to the subject a therapeutically effective amount at least one inhibitor of stress granule formation that reduces the expression of an immune checkpoint protein, wherein said step of administering enhances the proliferation, migration, persistence and/or activity of cytotoxic T lymphocytes (CTLs) in the subject.

3. The method of claim 2 wherein the immune checkpoint protein is PD-1.

4. A method of reducing T cell exhaustion in a subject in need thereof comprising administering to the subject a therapeutically effective amount of at least one inhibitor of stress granule formation sufficient to reduce T cell exhaustion in the subject.

5. The method of claim 1, wherein the subject suffers from a cancer.

6. The method of claim 5 wherein the cancer is selected from the group consisting of neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

7. The method of claim 5 wherein the cancer is characterized by a high tumor infiltration of cytotoxic T lymphocytes that express an immune checkpoint protein.

8. The method of claim 1, wherein the subject suffers from a viral infection.

9. The method of claim 1, wherein the inhibitor of stress granule formation inhibits the activity or expression of a kinase that is involved in the signaling pathway leading to the formation of stress granule wherein said kinase is selected from the group consisting of GCN2, PERK, PKR, HRI, mTOR, CK2, DYRK3, AMPK, ROCK1, S6K1, S6K2 and OGT.

10. The method of claim 9 wherein the inhibitor of stress granule formation is an inhibitor of activity or expression of GCN2 or PERK.

11. The method of claim 1, wherein the inhibitor of stress granule formation inhibits the activity or expression of a protein that is structurally involved in formation of stress granule wherein said protein is selected from the group consisting of ABCF1, ADAR, ADD1, AGO1, AG02, AHSA1, AKAP9, ALYREF, ANG, APOBEC3G, AQR, ATP2C1, ATXN2, ATXN2L, BCCIP, BRF1, CALR, CAPRIN1, CASC3, CCAR1, CCDC124, CCR4, CDC37, CELF1, CELF2, CIRBP, CNBP, CNOT8, CPEB1, CPEB2, CPEB3, CPEB4, CYFIP2, DAZAP1, DAZAP2, DAZL, DCP1A, DCP1B, DCP2, DDX1, DDX39A, DDX39B, DDX3X, DDX3Y, DDX5, DDX58, DDX6, DHX30, DHX33, DHX36, DHX58, DHX9, DROSHA, DYRK3, EDC3, EDC4, EIF2A, EIF2AK2, EIF2C1, EIF2S1, EIF2S2, EIF2S3, EIF3A, EIF3B, EIF3C, EIF3D, EIF3E, EIF3F, EIF3G, EIF3H, EIF3I, EIF3J, EIF3K, EIF3L, EIF3M, EIF4A1, EIF4A2, EIF4A3, EIF4B, EIF4E, EIF4G1, EIF4G2, EIF4G3, EIF4H, EIF5, EIF5A, EIF5A2, EIF5B, ELAVL1, ELAVL2, ELAVL3, ELAVL4, ETF1, EWSR1, FAM120A, FASTK, FMR1, FUBP1, FUBP3, FUS, FXR1, FXR2, G3BP1, G3BP2, GBP2, GIGYF2, GRB7, GSPT1, GSPT2, HDAC6, HNRNPA0, HNRNPA1, HNRNPA2B1, HNRNPA3, HNRNPAB, HNRNPC, HNRNPD, HNRNPH1, HNRNPK, HNRNPL, HNRNPM, HNRNPR, HNRNPU, HOPX, HSP90AA1, HSPA8, HSPB1, HSPD1, HTT, IGF2BP1, IGF2BP2, IGF2BP3, ILF2, ILF3, IP08, KHDRBS1, KHSRP, KPNA2, KPNA4, KPNA5, KPNB1, LARP4, LARP4B, LIN28A, LIN28B, LSM1, LSM12, LSM14A, LSM14B, MAP1LC3A, MAPK8, MATR3, MBNL1, MCRIP1, MCRIP2, METAP2, MEX3A, MEX3B, MSI1, MSI2, NCL, NELFE, NKRF, NOLC1, NONO, NPM1, NRG2, NUFIP2, NXF1, NXF5, OAS1, OAS2, OAS3, OGFOD1, OGG1, OGN, PABPC1, PABPC3, PABPC4, PABPC5, PAN2, PAN3, PARN, PATL1, PCBP1, PCBP2, PFN1, PFN2, PHB2, PKP1, PKP3, PNPT1, PPP1R8, PQBP1, PRKCA, PRKRA, PRMT1, PRRC2C, PSD3, PSPC1, PTBP1, PTK2, PUM1, PUM2, PURA, PURB, QKI, RACK1, RAN, RBM15, RBM17, RBM25, RBM3, RBM4, RBM42, RC3H1, RECQL, RHOA, RNASEL, RNH1, ROCK1, RPL3, RPS11, RPS18, RPS19, RPS24, RPS3, RPS6, RPS6KA3, RTCA, SAFB2, SAMD4A, SERBP1, SF1, SFPQ, SLBP, SMG1, SMN1, SMN2, SND1, SPATS2L, SRP68, SRSF5, SRSF7, SRSF9, STAU1, STAU2, SYNCRIP, TAF15, TARDBP, TDRD3, TIA1, TIAL1, TNPO1, TNRC6A, TNRC6B, TRAF2, TRIM2, TRIM3, TRIP6, TROVE2, UBAP2L, UPF1, UPF2, UPF3A, UPF3B, USP10, USP6, UTP18, WDR62, XRN1, XRN2, YBX1, YBX3, YTHDF1, YTHDF2, ZBP1, ZC3H11A, ZC3HAV1, ZFP36, and ZONAB.

12. The method of claim 1, wherein the inhibitor of stress granule formation is administered to the patient in combination with at least one immune checkpoint inhibitor.

13. An in vitro or ex vivo method of reducing expression of at least one immune checkpoint protein in a population of T cells comprising contacting the population of T cells with an amount of at least one inhibitor of stress granule formation sufficient to reduce the expression of the at least one immune checkpoint protein.

14. The method of claim 13 wherein the population of T cells is a population of T CD8+ cells, T CD4+ cells, or gamma delta T cells.

15. The method of claim 13 wherein the population of T cells is a population of CAR-T cells.

16. The method of claim 12, wherein the immune checkpoint inhibitor is a PD-1 antagonist, a PD-L1 antagonist, a PD-L2 antagonist, a CTLA-4 antagonist, a VISTA antagonist, a TIM-3 antagonist, a LAG-3 antagonist, a IDO antagonist, a KIR2D antagonist, a A2AR antagonist, a B7-H3 antagonist, a B7-H4 antagonist, or a BTLA antagonist.
